(19) United States
(12) Patent Application Publication
(10) Pub. No.: US 2025/0302709 A1
(43) Pub. Date: Oct. 2, 2025

(54) NANOPARTICLE SUNSCREEN COMPOSITIONS

(71) Applicant: EVOQ NANO, INC., Salt Lake City, UT (US)

(72) Inventor: William H. NIEDERMEYER, West Jordan, UT (US)

(21) Appl. No.: 19/094,511

(22) Filed: Mar. 28, 2025

Related U.S. Application Data
(60) Provisional application No. 63/571,200, filed on Mar. 28, 2024.

(51) Int. Cl. A61K 8/27; A61K 8/02; A61K 8/19; A61K 8/49; A61K 8/92; A61K 8/9789; A61Q 17/04

(52) U.S. Cl. CPC A61K 8/27; A61K 8/0245; A61K 8/025; A61K 8/19; A61K 8/498; A61K 8/922; A61K 8/9789; A61Q 17/04

(57) ABSTRACT

Sunscreen compositions for protecting against UV radiation include a carrier and a plurality of nonionic metal nanoparticles suspended within the carrier. The metal nanoparticles can be configured in size and shape so as to selectively scatter ultraviolet light incident upon the metal nanoparticles. The metal nanoparticles can be configured with a higher refractive index with respect to ultraviolet light than a refractive index with respect to visible light. Some metal nanoparticles can down-convert higher energy UV radiation to less harmless, lower energy visible light. The metal nanoparticles can include spherical-shaped and/or coral-shaped nanoparticles. The spherical-shaped nanoparticles can provide greater protection against UVB radiation than the coral-shaped nanoparticles, and the coral-shaped nanoparticles can provide greater protection against UVA radiation than the spherical-shaped nanoparticles.

### CROSS-REFERENCE TO RELATED APPLICATIONS

This application claims the benefit of U.S. Provisional Application No. 63/571,200, filed Mar. 28, 2024, which is incorporated by reference in its entirety.

### BACKGROUND

### 1. Field of the Invention

Disclosed are nanoparticle sunscreen compositions and methods for protecting skin against solar radiation, including compositions and methods for protecting against burns, cancer, and other damage to skin caused by ultraviolet (UV) radiation.

### 2. Relevant Technology

Exposure to solar radiation can cause or aggravate a variety of detrimental skin conditions, including burns, cancer, wrinkles, and pre-mature aging of the skin. In particular, ultraviolet radiation B (“UVB radiation”), which generally lies within the range of about 280 to about 315 nanometers in wavelength, can cause burning of the skin in cases of excessive exposure, while ultraviolet radiation A (“UVA radiation”), which generally lies within the range of about 315 to about 400 nanometers in wavelength, can cause faster aging of the skin in cases of excessive exposure. Both UVA and UVB radiation can cause cancer and/or DNA damage.

While many sunscreens are available, they do not always offer the sufficient levels of protection. In fact, sunscreens are typically rated with a sun protection factor (“SPF”), which is correlated with how much UVB radiation is blocked by the sunscreen. However, SPF ratings do not take into account skin damage caused by UVA rays, which do not primarily cause sunburns but can still contribute to cancer and skin aging. Further, some individuals are more susceptible to sunburning and sun damage, and a sunscreen which works for one individual may not offer sufficient protection for another with fairer or more sensitive skin.

In addition, typical sunscreens have active ingredients, such as benzenoid organic compounds, for absorbing radiation and reducing the amount that reaches the skin. Many of these compounds are known to cause eye and/or skin irritation in some individuals, and many are known to be or are thought to possibly be endocrine disruptors and/or carcinogens. Further, such sunscreen ingredients tend to break down during exposure to sunlight, necessitating frequent reapplication.

“Sunblocks” are an alternative to chemical sunscreens and utilize titanium dioxide or zinc oxide particulates to block and/or reflect incident light. These compounds are typically mixed into a thick carrier oil to prevent being washed off the skin. Sunblocks typically have a thick and pasty consistency to keep them from easily washing off, which can make them difficult to spread over the entirety of the body where coverage is desired. In addition, such sunblocks are opaque and can be seen on the skin after application, which can be aesthetically unpleasing. Further, some of these compounds (such as titanium dioxide) have photocatalytic activity that can produce reactive oxygen species (ROS) and other free radicals, particularly in the presence of solar radiation and moisture, which are common when using sunscreen. ROS and other free radicals can lead to skin damage and other detrimental health effects.

Accordingly, there has been and remains a need to find beneficial sunscreen compositions and methods for protecting skin from burns, disease, and damage caused by UV radiation, and which are safer than chemical sunscreens and easier to use and more pleasant feeling than sunblocks.

### SUMMARY

Disclosed herein are nanoparticle sunscreen compositions and methods for protecting skin or other exposed materials from ultraviolet (UV) radiation. In some embodiments, a method of protecting skin from UV radiation comprises: (1) applying a nanoparticle sunscreen composition comprising a carrier and metal nanoparticles onto a treatment area, (2) the nanoparticle sunscreen composition reflecting and/or down-converting (shifting to lower, less harmful wavelengths) at least a portion of the UV light incident upon the treatment area.

In some embodiments, metal nanoparticles can comprise spherical-shaped metal nanoparticles and/or coral-shaped metal nanoparticles. In some embodiments the coral-shaped metal nanoparticles can be used together with spherical-shaped metal nanoparticles (e.g., in order to potentiate beneficial effects of the spherical-shaped metal nanoparticles and/or maintain them in solution or as a well-dispersed suspension).

In some embodiments, nanoparticle sunscreen compositions, such as single- or multi-component nanoparticle sunscreen compositions, include a stabilizing agent capable of maintaining the nanoparticles well-dispersed in solution and limiting or preventing agglomeration while maintaining functionality of the nanoparticles.

Embodiments disclosed herein can provide a variety of advantages and benefits. For example, at least some embodiments of nanoparticle sunscreen compositions can provide broad spectrum protection against both UVA and UVB radiation without irritating the skin and/or can provide high stability, thereby lessening the amount of reapplication otherwise required to maintain effectiveness. In some embodiments, nanoparticle sunscreen compositions can be provided in a visibly transparent and easily applied form. The compositions can have active ingredients that are not absorbable or have low absorption into skin and/or bloodstream of a user and that do not contain endocrine disruptors or carcinogens. The compositions can have active ingredients that cause little or no photocatalytic production of ROS and other free radicals.

In some embodiments, nanoparticle sunscreen compositions can provide wavelength shifting capabilities to down-convert shorter, more energetic wavelengths to longer, less energetic wavelengths. For example, spherical-shaped gold nanoparticles having a particle size less than about 40 nm (e.g., 4-20 nm) are able to down-convert incoming UV radiation to a longer wavelength (lower frequency) and less harmful radiation. In some embodiments, spherical-shaped gold nanoparticles can down-convert incoming UV radiation by up to about 200 nm. Beneficially, this down-converting of wavelengths (frequencies) can limit or prevent sunburns or other skin damage caused by UV radiation.

These and other advantages and features of the invention will be set forth in part in the description which follows, and in part will become apparent to those skilled in the art upon examination of the following or may be learned by practice of the invention.

### BRIEF DESCRIPTION OF THE DRAWINGS

FIGS. 1A-1D are transmission electron microscope (TEM) images of various non-spherical-shaped nanoparticles (i.e., that have surface edges and external bond angles) made according to conventional chemical synthesis or electrical discharge methods;

FIGS. 2A-2C are TEM images of spherical-shaped metal nanoparticles (i.e., that have no surface edges or external bond angles), the nanoparticles having substantially uniform size and narrow particle size distribution, smooth surface morphology, and solid metal cores and that are devoid of coating or capping agents, and that have a high degree of sphericity and roundness for use in making nanoparticle sunscreen compositions;

FIGS. 3A-3C are TEM images of coral-shaped nanoparticles for use in making nanoparticle sunscreen compositions;

FIGS. 4A and 4B schematically compare the sphericities of a spherical-shaped nanoparticle made by laser ablation and a hedron-shaped nanoparticle made by chemical synthesis based on a comparison of the aspect ratios;

FIGS. 5A and 5B schematically compare the sphericities of hypothetical nanoparticles based on a comparison of the volume equivalent sphere and the surface area of the actual nanoparticle; and

FIGS. 6A and 6B are TEM images that illustrate and compare the circularities of a spherical-shaped nanoparticle made by laser ablation and a typical colloidal silver nanoparticle made by chemical synthesis.

### DETAILED DESCRIPTION

Disclosed herein are nanoparticle sunscreen compositions and methods for preventing sunburns, cancer, premature aging, and other skin damage caused by exposure to UV radiation. Also disclosed are methods for making and using such nanoparticle sunscreen compositions.

As used herein, the terms “sunscreen,” “sunscreen composition,” and “nanoparticle sunscreen composition” refer to compositions that include a plurality of nonionic metal nanoparticles made by laser ablation and a carrier. The compositions are configured to protect against UV radiation at least in part by the blocking, scattering, reflecting, and/or wavelength down-converting by the metal nanoparticles (i.e., via plasmon resonance). Similar terms, such as “sunblock,” “sun cream,” “suntan lotion,” “tanning cream,” “tanning oil,” and other such compositions are also included within the definition.

In some embodiments, at least two types of nanoparticles (e.g., that differ in size, shape, and/or chemistry) can be utilized in the disclosed sunscreen compositions, each with specific particle size distribution, and a stabilizing agent (such as natural-based polyphenol, cream, gel, or surfactant) to stabilize the nanoparticle components and which may also be beneficial for use in dermal and/or sunscreen applications. Such compositions can effectively protect a treated area from burns and other skin damage resulting from exposure to UV radiation.

### Nanoparticle Configurations

In some embodiments, the metal nanoparticles may comprise, consist or consist essentially of nonionic, ground state metal nanoparticles. Examples include spherical-shaped metal nanoparticles, coral-shaped metal nanoparticles, or a blend of spherical-shaped and coral-shaped metal nanoparticles.

In some embodiments, metal nanoparticles useful for making nanoparticle compositions comprise spherical-shaped nanoparticles having a solid core and no organic or other capping agents or cores. The term “spherical-shaped metal nanoparticles” refers to nanoparticles that are made from one or more metals, preferably nonionic, ground state metals, having only internal bond angles and no external edges or bond angles. In this way, the spherical-shaped nanoparticles are highly resistant to ionization, highly stable, and highly resistance to agglomeration. Such nanoparticles exhibit a high ξ-potential, which permits the spherical-shaped nanoparticles to remain dispersed within a polar solvent without a surfactant.

Examples of methods and systems for manufacturing spherical-shaped metal nanoparticles by laser ablation or electric discharge to form an initial plume of nanoparticles, coupled with cross-laser manipulation of nanoparticle size, are disclosed in U.S. Pat. Nos. 9,849,512, 10,137,503, and 10,610,934 to William Niedermeyer, which are incorporated herein by reference in their entirety.

It should be understood that the spherical-shaped metal nanoparticles made according to the Niedermeyer patents differ substantially from conventional colloidal silver or other metal nanoparticles formed by chemical or other processes that yield ionic solutions and/or colloidal metal particles with crystal facets and edges that release metal ions. They especially differ from metal nanoparticles that have organic capping agents and/or organic or other non-metallic cores and which have significantly lower sphericity and roundness compared to spherical-shaped nanoparticles made by laser ablation disclosed herein.

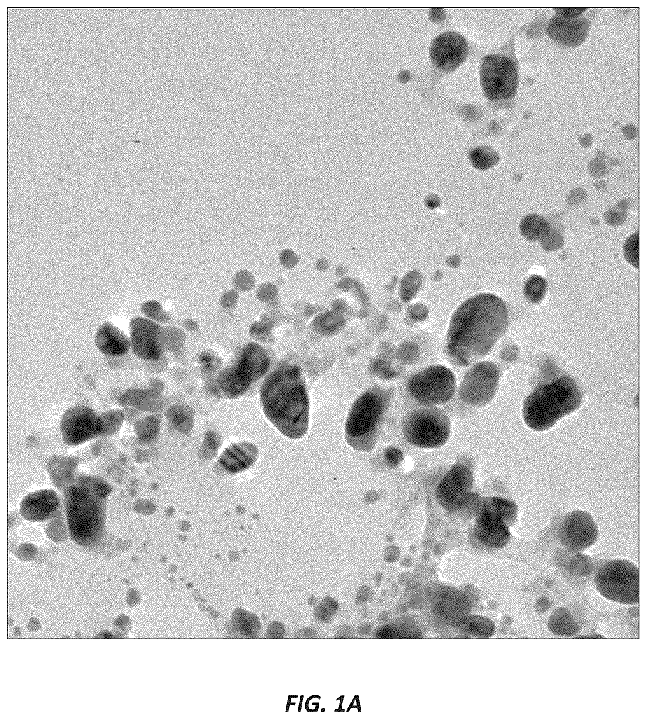

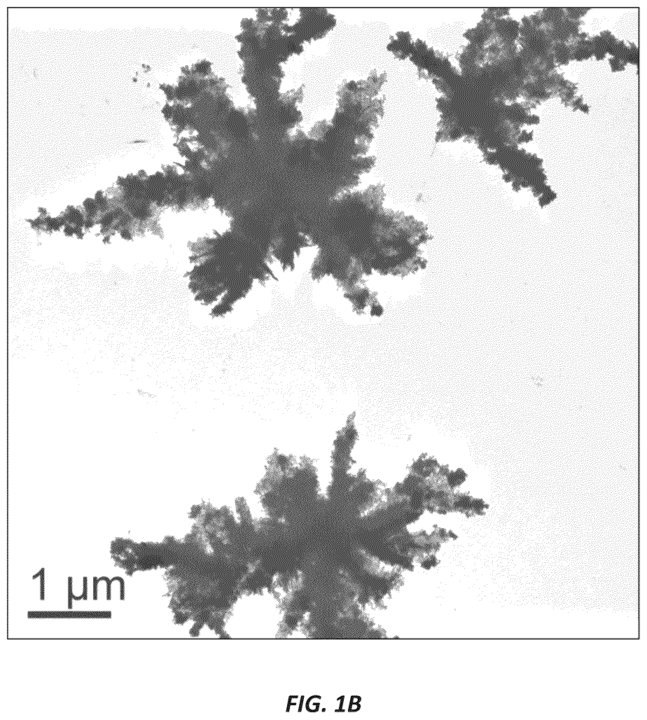

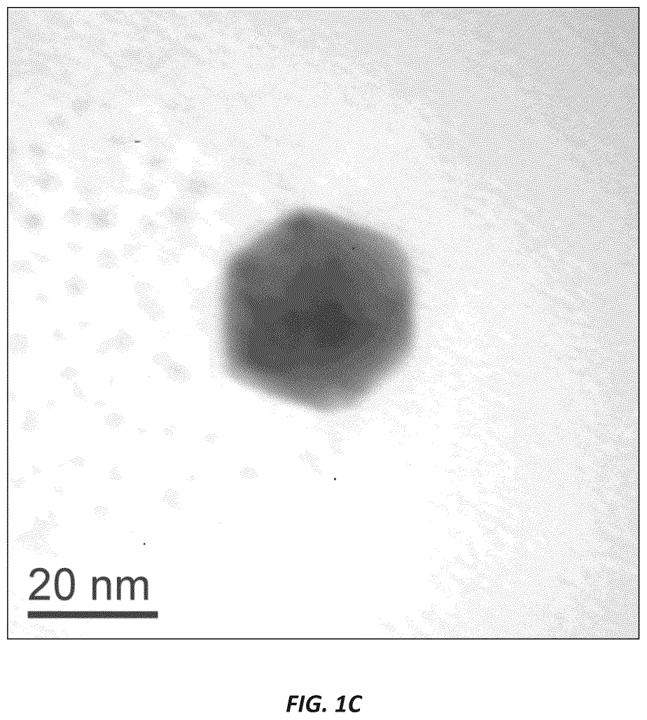

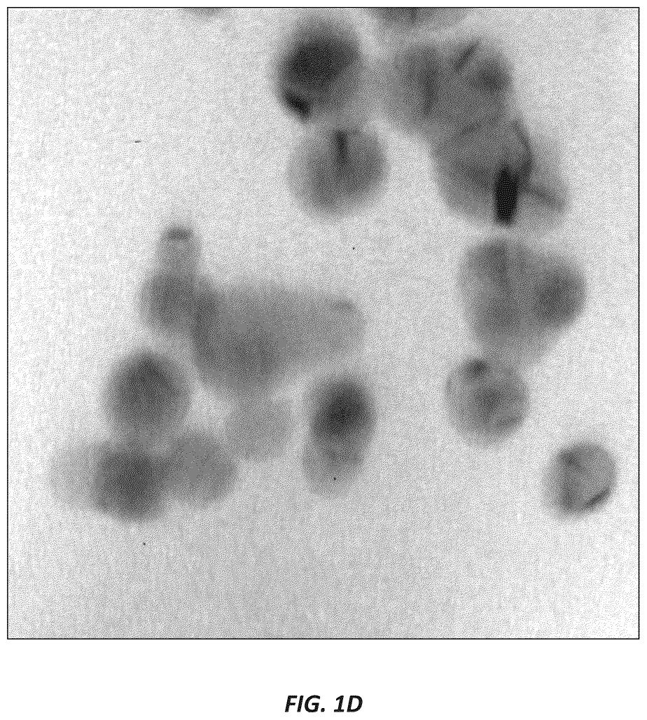

For comparison purposes, FIGS. 1A-1D are transmission electron microscope (TEM) images of nanoparticles made according to various chemical synthesis methods. As shown, the nanoparticles formed using these synthesis methods tend to exhibit a clustered, crystalline, faceted, and/or hedron-like shape rather than a true spherical shape with round and smooth surfaces. Even so-called “spherical” nanoparticles made by chemical synthesis methods are not truly spherical and typically have significantly lower sphericity and roundness compared to a perfect sphere, which has a sphericity and roundness of 1.

For example, FIG. 1A shows silver nanoparticles formed using a common trisodium citrate method. The nanoparticles tend to be clustered (agglomerated), have a broad particle size distribution (i.e., the difference in particle size between the largest particles, smallest particles, and mean particle size is very large), and a sphericity and roundness that deviate significantly from 1 even in an un-agglomerated state. FIG. 1B shows another type of silver nanoparticles (available from American Biotech Labs, LLC) formed using another chemical synthesis method. Such nanoparticles have rough surface morphologies with many edges and are sometimes referred to as “nanoflowers” and very low sphericity and roundness. They are jagged with numerous edges or crystal facets. FIG. 1C shows a gold nanoparticle having a hedron shape (hexagonal in cross section) rather than a true spherical shape (although such particles are often called “spherical”, they are not “spherical” as that term is defined herein, but have a sphericity and roundness that deviate significantly from 1). FIG. 1D shows silver nanoparticles (sold under the trade name MesoSilver) that have relatively smoother surface morphologies but are understood to be shells of silver formed over seeds of non-metallic core material. They have a sphericity and roundness that deviate significantly from 1.

In contrast, the spherical-shaped nanoparticles described herein and incorporated into nanoparticle sunscreen compositions are solid metal, substantially unclustered, exposed, uncoated, and uncapped, and have a smooth and round surface morphology along with a narrow size distribution and a sphericity and roundness that approach or equal 1.

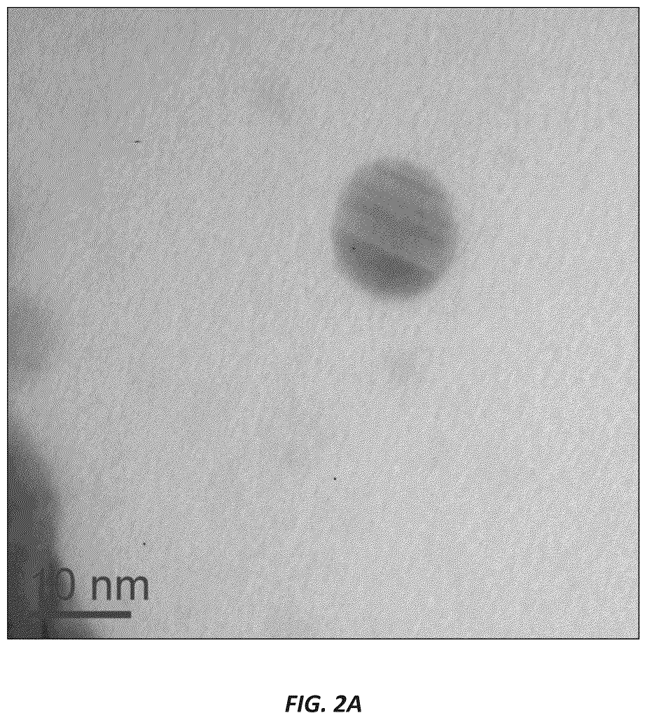

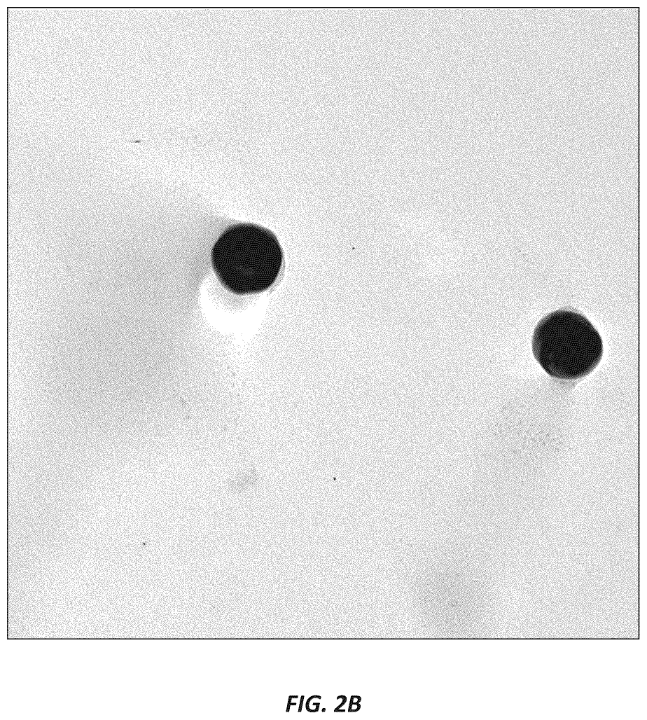

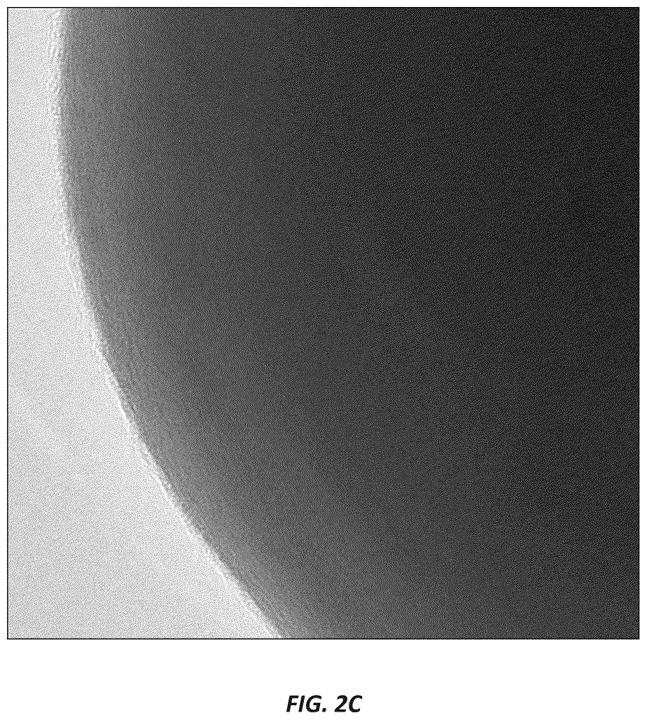

FIGS. 2A-2C are TEM images of spherical-shaped metal nanoparticles that can be used herein. FIG. 2A shows a gold/silver alloy nanoparticle (90% silver and 10% gold by molarity) having a sphericity and roundness that approach or equal 1. FIG. 2B shows two spherical-shaped nanoparticles side by side to visually illustrate size similarity. FIG. 2C shows the surface of a metal nanoparticle showing the smooth and edgeless surface morphology devoid of crystal facets found in conventional colloidal silver or other metal nanoparticles, which a sphericity and roundness that approach or equal 1. The smooth surface prevents the release of metal ions compared to traditional colloidal silver, which is ionic and has external bond angles that promote ionization. Preferred spherical-shaped metal (e.g., gold or silver) nanoparticles have a sphericity and roundness of at least 0.99 and that approach or equal 1.

In some embodiments, spherical-shaped metal nanoparticles can have a diameter of about 40 nm or less, about 35 nm or less, about 30 nm or less, about 25 nm or less, about 20 nm or less, about 15 nm or less, about 10 nm or less, about 7.5 nm or less, or about 5 nm or less. In a preferred embodiment, the spherical-shaped metal nanoparticles comprise spherical-shaped (e.g., gold) nanoparticles having a particle size in range of about 4 nm to about 20 nm, or about 6 nm to about 15 nm, or about 8 nm to about 12 nm.

In some embodiments, spherical-shaped metal nanoparticles can have a mean diameter and a particle size distribution such that at least 99% of the nanoparticles have a diameter within 30% of the mean diameter, or with 20% of the mean diameter, or within 10% of the mean diameter. In some embodiments, spherical-shaped nanoparticles can have a mean particle size and a particle size distribution such that at least 99% of the nanoparticles have a particle size that is within +3 nm of the mean diameter, +2 nm of the mean diameter, or +1 nm of the mean diameter. The mean diameter of spherical-shaped metal nanoparticles can be determined by dynamic light scattering using intensity-weighted average.

In some embodiments, spherical-shaped nanoparticles can have a ¿-potential of at least ±10 mV (absolute value), preferably at least about +15 mV, more preferably at least about ±20 mV, even more preferably at least about ±25 mV, and most preferably at least about ±30 mV.

In some embodiments, nonionic metal nanoparticles useful for making nanoparticle sunscreen compositions may comprise coral-shaped nanoparticles or addition to or in place of spherical-shaped nanoparticles. The term “coral-shaped metal nanoparticles” refers to nanoparticles that are made from one or more metals, preferably nonionic, ground state metals having a non-uniform cross section and a globular structure formed by multiple, non-linear strands joined together without right angles. They have low sphericity and roundness similar to colloidal silver and other non-spherical nanoparticles. Similar to spherical-shaped nanoparticles, coral-shaped nanoparticles have only internal bond angles and no external edges or bond angles. In this way, coral-shaped nanoparticles are highly resistant to ionization, highly stable, and highly resistant to agglomeration. Such coral-shaped nanoparticles can exhibit a high ¿-potential similar to spherical-shaped nanoparticles, which permits the coral-shaped nanoparticles to remain dispersed within a polar solvent without a surfactant.

In some embodiments, coral-shaped nanoparticles can have a length ranging from about 15 nm to about 100 nm, or about 25 nm to about 95 nm, or about 40 nm to about 90 nm, or about 60 nm to about 85 nm, or about 70 nm to about 80 nm. In some embodiments, coral-shaped nanoparticles have a mean particle size and a particle size distribution such that at least 99% of the nanoparticles have a length within 30% of the mean length, or within 20% of the mean length, or within 10% of the mean length. In some embodiments, coral-shaped nanoparticles can have a ¿-potential of at least ±10 mV (absolute value), preferably at least about ±15 mV, more preferably at least about ±20 mV, even more preferably at least about ±25 mV, and most preferably at least about ±30 mV.

Examples of methods and systems for manufacturing coral-shaped metal nanoparticles by laser ablation or electric discharge to form an initial plume of nanoparticles, coupled with cross-laser manipulation of nanoparticle size, are disclosed in U.S. Pat. No. 9,919,363 to William Niedermeyer, which is incorporated by reference in its entirety.

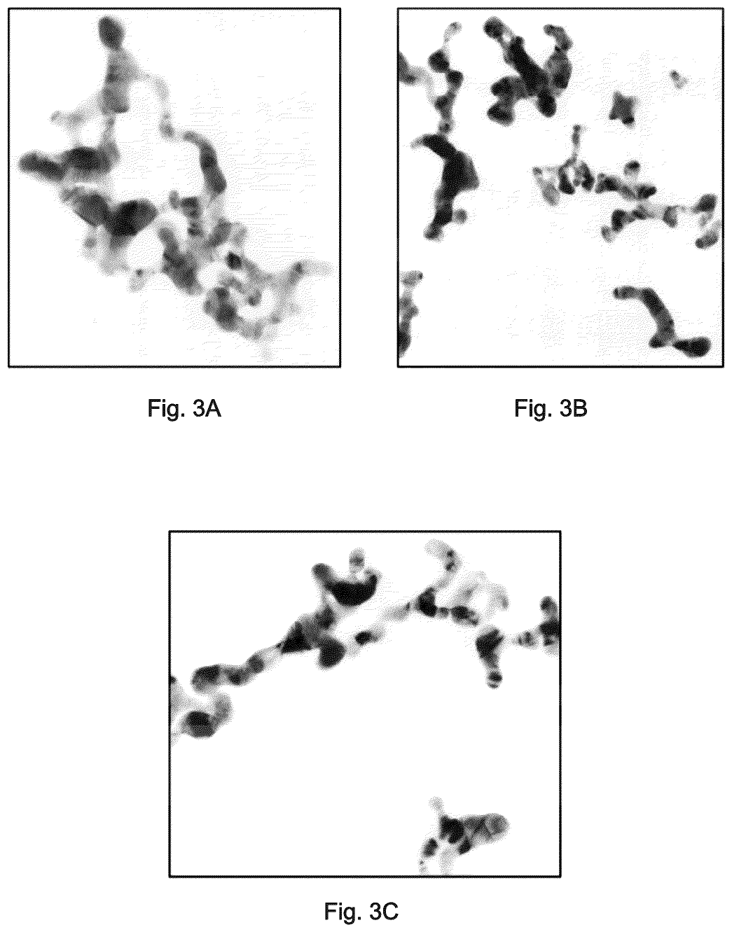

FIGS. 3A-3C are transmission electron microscope images (TEMs) of exemplary coral-shaped gold nanoparticles. Coral-shaped metal nanoparticles are not “nanoflowers” and have no physical or chemical resemblance to nanoflowers known in the art (which have edges and external bond angles).

The metal nanoparticles, including spherical-shaped and coral-shaped nanoparticles, may comprise any desired metal, mixture of metals, or metal alloy, including at least one of silver, gold, platinum, palladium, rhodium, osmium, ruthenium, rhodium, rhenium, molybdenum, copper, iron, nickel, tin, beryllium, cobalt, antimony, chromium, manganese, zirconium, zinc, tungsten, titanium, vanadium, lanthanum, cerium, heterogeneous mixtures thereof, or alloys thereof.

In preferred embodiments, the metal nanoparticles are formed from material(s) and/or are configured in size and shape to limit or eliminate photocatalytic activity that result in the production of ROS and/or other free radicals. In addition, the metal nanoparticles are preferably formed from material(s) that minimize or eliminate the potential for irritation or discomfort (e.g., skin irritation) upon application. For example, in preferred embodiments, the metal nanoparticles may be formed from one or more of gold, silver, zinc, titanium, iron, copper, cobalt, chromium, or manganese. In more preferred embodiments, the metal nanoparticles may be formed form one or more of gold, zinc, titanium, and silver.

### Sphericity

The spherical-shaped metal nanoparticles disclosed herein are characterize as having high sphericity, approaching or equaling a sphericity of 1, which defines a perfect sphere. Sphericity can affect how metal nanoparticles behave, particularly when the particle size is below about 20 nm. The changes in behavior of metal nanoparticles below about 20 nm differ depending on morphology, such that the behavior of a quasi sphere or a facetted sphere is different than that of a smooth sphere. This can be seen in zeta potential differences due to point charges where facets meet and chemical interaction with surface area. Also, plasmon resonance can be affected by irregular shapes in comparison to round smooth spheres.

To illustrate the geometric differences between spherical shaped metal nanoparticles (e.g., EVQ-218 nano spheres, which are available from Evoq Nano, located in Salt Lake City, Utah) and equivalent synthesized silver nano colloids, reference is made to math and imaging data below. One way to determine the sphericity of a nanoparticle is to determine the ratio of surface morphology by its maximum diameter and minimum diameter. While Transmission Electron Microscopy (TEM) only shows two dimensions of the nanoparticles, the ratio of the maximum and minimum diameters of a metal nanoparticle is one way to determine its sphericity.

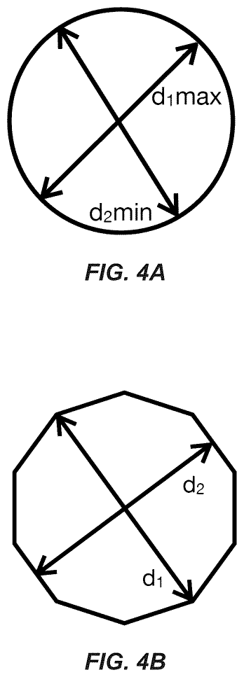

Reference is made to FIGS. 4A and 4B, which compare the ratios of the maximum and minimum diameters of a spherical metal nanoparticle and a faceted, quasi spherical metal nanoparticle. FIG. 4A schematically illustrates the maximum diameter (d₁max) and minimum diameter (d₂min) of a smooth spherical-shaped metal nanoparticle. In this case, the maximum and minimum diameters are the same, with an aspect ratio of 1:1 or 100% sphericity (d1=d2), (sphericity of 1). FIG. 4B schematically illustrates the maximum diameter (d₁max) and minimum diameter (d₂min) of a faceted, quasi spherical metal nanoparticle. In this case, the maximum and minimum diameters are not the same. Instead, the maximum diameter (d1) is larger than the minimum diameter (d2), such that the aspect ratio is greater than 1 (d1>d2). In this case, the aspect ratio is 1:0.94 or a sphericity of 0.94.

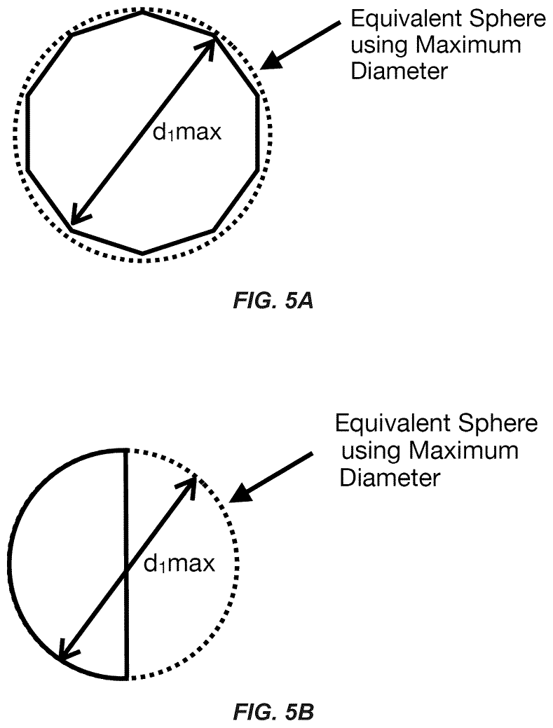

Reference is made to FIGS. 5A and 5B, which illustrate sphericity in terms of the volume equivalent of a sphere with the surface area of the actual particle. In a perfect sphere, this ratio again is 1:1, which equates to a sphericity of 1. However, in an irregular or quasi sphere, they are not equal. FIG. 5A schematically illustrates a hedron-shaped quasi spherical nanoparticle in which the maximum diameter (d₁max) of the nanoparticle is equal to the diameter of the circumscribing sphere but where the facets do not extend to circumference such that the nanoparticle has a sphericity of less than 1. FIG. 5B schematically illustrates an extreme example of a smooth nanoparticle hemisphere with a low sphericity that is half the volume sphere, or a sphericity of 0.5.

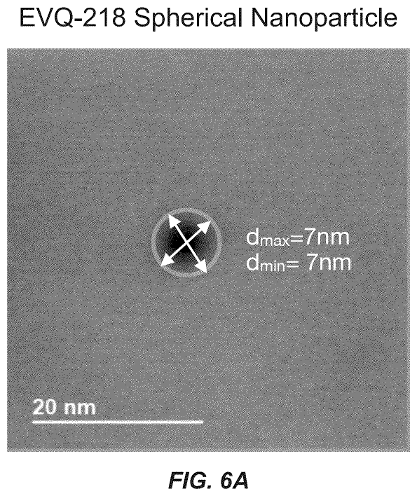

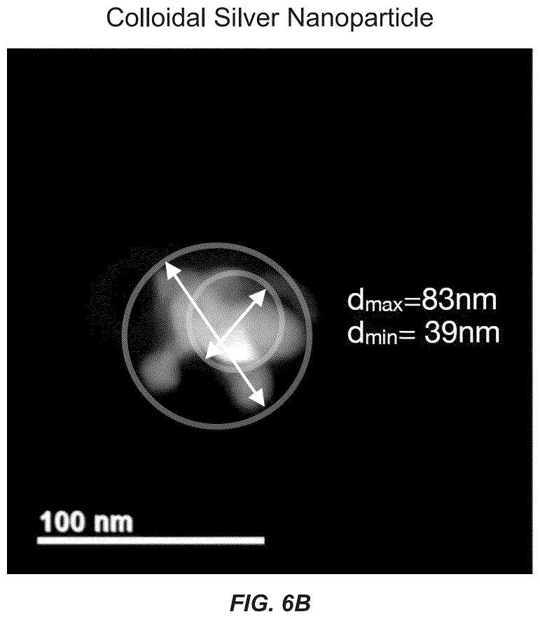

Reference is made to FIGS. 6A and 6B, which are TEM images that illustrate and compare the 2-dimensional circularities of a spherical-shaped nanoparticle made by laser ablation and a typical colloidal silver nanoparticle made by chemical synthesis. Circularity is similar to the ratios discussed above relative to FIGS. 4A-4B and 5A-5B, but based on the square root of the diameter aspect ratio (dmax/dmin)^{1/2}. FIG. 6A is a TEM image of a spherical-shaped EVQ-218 silver nanoparticle, which has a maximum diameter of 7 nm (dmax=7 nm) and minimum diameter of 7 nm (dmin=7 nm). The aspect ratio is therefore 1, with the sphericity being 1 and the circularity also being 1. Thus, spherical-shaped metal nanoparticles made by laser ablation as disclosed herein have a very high sphericity that approaches or equals 1, a high circularity that approaches or equals 1, and an aspect ratio that approaches or equals 1. FIG. 6B, by contrast, is a TEM image of a typical colloidal silver nanoparticle made by chemical synthesis, which has a maximum diameter of 83 nm (dmax=83 nm) and a minimum diameter of 39 nm (dmin=39 nm). The colloidal silver nanoparticle has a sphericity that is complex due to irregular morphology. However, the aspect ratio, which is the inverse of sphericity, is 89 nm/39 nm (or 2.28) and the square root of the aspect ratio, which is the inverse of circularity, is 1.51.

### Multi-Component Nanoparticle Compositions

In some embodiments, coral-shaped metal nanoparticles can be used in combination with spherical-shaped metal nanoparticles. In general, spherical-shaped metal nanoparticles can be smaller than coral-shaped metal nanoparticles and in this way can provide very high surface area for catalyzing desired reactions or providing other desired benefits. On the other hand, the generally larger coral-shaped nanoparticles can exhibit higher surface area per unit mass compared to spherical-shaped nanoparticles because coral-shaped nanoparticles have internal spaces and surfaces rather than a solid core and only an external surface. Nanoparticle compositions that contain a mixture of spherical-shaped and coral-shaped metal nanoparticles are disclosed in U.S. Pat. No. 9,434,006 to William Niedermeyer, which is incorporated by reference in its entirety.

In some cases, providing nanoparticle compositions containing both spherical-shaped and coral-shaped nanoparticles can provide synergistic results. In most cases, the main purpose of coral-shaped nanoparticles is to maintain spherical-shaped nanoparticles in solution and preventing them from agglomerating. Coral-shaped nanoparticles may help carry and/or potentiate the activity of spherical-shaped nanoparticles in addition to providing their own unique benefits. In some embodiments, a combination of spherical-shaped and coral-shaped nanoparticles can lead to synergistic, broad-spectrum protection with a greater amount of protection (e.g., amount of UV radiation reflected and/or down converted) per amount of nanoparticles relative to single sized and/or shaped nanoparticle compositions.

In some embodiments, such as where the carrier is a hydrophilic liquid, such as water and/or alcohol, the mass ratio of spherical-shaped nanoparticles to coral-shaped nanoparticles in the nanoparticle composition can be in a range of about 1:1 to about 50:1, or about 2.5:1 to about 25:1, or about 5:1 to about 20:1, or about 7.5:1 to about 15:1, or about 9:1 to about 11:1, or about 10:1. The particle number ratio of spherical-shaped nanoparticles to coral-shaped nanoparticles in the nanoparticle composition can be in a range of about 10:1 to about 500:1, or about 25:1 to about 250:1, or about 50:1 to about 200:1, or about 75:1 to about 150:1, or about 90:1 to about 110:1, or about 100:1.

In other embodiments, such as where the carrier comprises a hydrophobic material or gelling agent that may cause spherical-shaped metal nanoparticles to agglomerate and precipitate from the carrier, and where the carrier does not include a stabilizing agent, the mass ratio of coral-shaped nanoparticles to spherical-shaped nanoparticles can be in a range of greater than 1:1 to about 50:1, or about 1.5:1 to about 25:1, or about 2:1 to about 15:1, or about 3:1 to about 10:1. In some embodiments, the mass ratio of coral-shaped nanoparticles to spherical-shaped nanoparticles can be at least about 0.75:1, 1:1, 1.25:1, 1.5:1, 1.75:1, 2:1, 2.5:1, 3:1, 4:1. 5:1. 6:1, 8:1, 10:1, 12:1 or 15:1 and less than about 100:1, 80:1, 60:1, 50:1, 40:1, 35:1, 30:1, 25:1 or 20:1, or within a range with endpoints of any two of the foregoing ratios. The coral-shaped metal nanoparticles can form a matrix in the carrier that is attractive to, but does not cause agglomeration of, spherical-shaped metal nanoparticles in order to maintain high dispersion and prevent agglomeration and precipitation of spherical-shaped metal nanoparticles from the carrier.

In some embodiments, a sunscreen composition may comprise (1) a set of spherical-shaped metal nanoparticles having a particle size and particle size distribution, (2) optionally a second set of metal nanoparticles having a second particle size and particle size distribution, (3) a stabilizing agent, and (4) a carrier. The carrier may comprise a stabilizing agent and/or may comprise one or more other components for delivery of the multicomponent nanoparticles onto the treatment area (e.g., portion of skin where UV protection is desired) of a person or animal.

In some embodiments, at least one of the first or second set of metal nanoparticles is selected so as to selectively reflect, block, scatter and/or down-convert a particular range of solar radiation. For example, the first set of metal nanoparticles may be spherical-shaped metal nanoparticles having a smaller relative size and sphericity and roundness that approach or equal 1 and which selectively or more particularly reflect, block, scatter and/or down-convert UVB and UVB radiation, and the second set of metal nanoparticles may be coral-shaped metal nanoparticles having a larger relative size and which help stabilize and maintain the spherical-shaped nanoparticles in solution or stable suspension. In other embodiments, the first and second sets of nanoparticles may both be spherical-shaped or may both be coral-shaped, but have different sizes and/or size distributions.

In some embodiments, the compositions may include a smaller spherical-shaped nanoparticle component and a larger coral-shaped nanoparticle component. In such embodiments, the spherical-shaped metal nanoparticles can be present in the composition in a range of about 100 ppb to about 50 ppm, or about 500 ppb to about 25 ppm, or about 1 ppm to about 15 ppm, or about 1 ppm to about 5 ppm. The larger coral-shaped nanoparticles can be present in the composition in a range of about 50 ppb to about 25 ppm, or about 200 ppb to about 15 ppm, or about 500 ppb to about 5 ppm, or about 1 ppm to about 3 ppm. It should be understood that the upper concentration range endpoints may not reflect an upper efficacy limit but a practical cost limit. Thus, in other embodiments, the spherical-shaped nanoparticles may present at a concentration above 5 ppm, or above 15 ppm, or above 25 ppm, or above 50 ppm and/or the coral-shaped nanoparticles may be present at a concentration above 3 ppm, or above 5 ppm, or above 15 ppm, or above 25 ppm.

### Stabilizing Agents

In some embodiments, the metal nanoparticle composition may include a stabilizing agent. For example, it may be desirable to have different specifically sized nanoparticles within the same solution in order to take advantage of each of the different properties and effects of the different particles. However, when differently sized metal nanoparticles are mixed into a single solution, the overall long-term stability of these particles within that single solution may be substantially diminished as a result of unequal forces exerted on the various particles causing eventual agglomeration of the nanoparticles. This may be more pronounced when the solution is heated or cooled significantly above or below standard room temperature conditions.

The stabilizing agent may itself be beneficial for use in dermal and/or sunscreen applications. Examples of stabilizing agents include alcohols (e.g., ethanol, propanol, butanol, etc.), as alcohols have been observed to effectively maintain nanoparticles of different sizes and different shapes within a given solution. Other examples of stabilizing agents include polyphenols (e.g., natural-based polyphenols such as arjuna bark extract, grape seed extract, etc.), which can have particular advantages in dermally applied sunscreen applications. Other examples include triglycerides such as grape seed oil, coconut oil, and the like, and other oils such as lavender and other terpenes. Yet other examples include amine compounds such as mono-, di-, and triethanol amine, and carbohydrates such as sucrose, fructose, and higher polymers, which have the ability to stabilize single- or multi-component nanoparticle compositions.

Stabilizing agents such as natural-based polyphenols (which include compounds such as grape seed oil, grape seed extract (e.g., the water soluble portion), arjuna bark extract, ethanolamines, or any other water soluble polyphenol sources and the like), can be dissolved into a carrier (e.g., water, alcohol, water-alcohol combination, or any combination of other liquid phase materials readily absorbed into the dermal region of a person or animal). Natural-based polyphenols typically show good efficacy when dissolved within a carrier in micro-to milli-molar concentrations ranges, with the upper range limitation typically being constrained not by efficacy but by cost.

Additional examples of stabilizing agents include liposomes, creams, and other emulsions. These and similar examples can stabilize the multi-component nanoparticle compositions while constituting the majority of the overall composition, which may contain little or no free water, alcohol or other liquid-phase components.

### Carriers

Given the ability of stabilizing agents to readily dissolve into water, alcohols and/or oils, introduction or manufacture of nanoparticles in solution with the stabilizing agents allows the nanoparticle compositions to be readily incorporated into any number of carriers, which can then be incorporated into a wide array of sunscreen products including sprays, creams, powders, gels, lotions, oils, emulsions, or jellies. In some embodiments, the metal nanoparticles may be incorporated into a carrier that is a cosmetic composition or may be incorporated into an ingredient that is included in a cosmetic composition. For example, nanoparticle compositions may be incorporated into a carrier that forms or is part of makeup (e.g., primer, concealer, foundation, powder, blush, etc.), lipstick, lip balm, and the like.

In some embodiments, the nanoparticle composition may include a separate stabilizing agent and a carrier. In other embodiments, the stabilizing agent may itself function as a carrier for delivering metal nanoparticles onto skin to be protected from solar radiation. The carrier can be a liquid, gel, or solid. Some carriers may be more suitable than others depending on the desired level of sun protection, desired texture, desired water-resistance, etc. For example, a carrier in the form of a cream or lotion may be used to provide the sunscreen composition in a form that can be applied for preventing sunburns and other damage, while a carrier in the form of an oil or spray may be used to provide the sunscreen composition in a form that can be applied for gradual tanning (e.g., by intentionally lowering the concentration of nanoparticles and/or by altering the composition of the nanoparticles).

Examples of compounds that can be utilized for sunscreen applications and as carriers to formulate nanoparticle compositions in accordance to the present invention, either in conjunction with one or more separate stabilizing agents or as the stabilizing agent, include, but are not limited to, water, alcohols, ketones, esters, citrus oils, essential oils, vegetable and other plant and natural oils, triglycerides, ethers, organic solvents, methanol, ethanol, isopropyl alcohol, other alcohols, glycols, glycerin, polyols, 1,3-propandiol, petroleum jelly, waxes, polymers, polymerizable materials, and surfactants.

In a preferred embodiment, the carrier may be a cream or lotion including a glycerin and/or stearic acid cream base optionally containing oils such as coconut oil, olive oil, grape seed oil, shea butter, mango butter, and/or vitamin E oil along with an emulsifying wax. Such a carrier composition can also act as the stabilizing agent to maintain the nanoparticle composition within the cream or lotion.

In another preferred embodiment, the carrier can be a water or combined water and alcohol solution, which contains a micro-to millimolar concentration of a separate stabilizing agent dissolved in the carrier to stably maintain the nanoparticles within the overall composition.

Gels known in the art can be used as carriers, such as gels containing one or more of the foregoing liquid components together with gelling agents known in the art. In some embodiments, gel compositions can more easily adhere to an area of the skin to which the composition is applied.

In some embodiments, a nanoparticle sunscreen composition can be formulated so that the metal nanoparticles are included at a concentration so that a measured quantity of the nanoparticle composition, when applied onto the skin, will provide a predetermined concentration or quantity of metal nanoparticles and/or will provide continuous protection from UV radiation for an extended period of time. Depending on the skin or treatment site, the nature of the nanoparticles being added, and the type of carrier being used, the nanoparticle composition may contain about 250 ppb to about 100 ppm of metal nanoparticles by weight, or about 500 ppb to about 50 ppm, or about 1 ppm to about 25 ppm, or about 3 ppm to about 20 ppm metal nanoparticles by weight.

In some embodiments, the nanoparticle composition can also include one or more optional components or adjuvents to provide desired properties, including, but not limited to, skin conditioners, plant extracts (e.g., aloe, arjuna bark extract, grape seed extract), astringents (e.g., witch hazel), moisturizers, emollients, antiseptics, lanolin, citric acid, Vitamin C, Vitamin E, fragrance, photostabilizers, self-tanning agents, etc.

### Sunscreen Activity

In some embodiments, the sunscreen compositions can include nanoparticles having a high refractive index in order to reflect and/or scatter incident UV radiation. For example, nanoparticles and/or multi-component nanoparticles used in sunscreen compositions of the present disclosure can have a refractive index for UVA and/or UVB radiation of about 1.5 to about 4.6, or from about 2.0 to about 4.0, or from about 2.5 to about 3.5. In some embodiments, the refractive index of the nanoparticles and/or multi-component nanoparticles will be higher relative to UVB radiation than UVA radiation (e.g., the refractive index increases with decreasing wavelength). In other embodiments, the refractive index of the nanoparticles and/or multi-component nanoparticles may be lower with respect to UVB radiation relative to UVA radiation (e.g., the refractive index increases with increasing wavelength). In general, smaller (e.g., spherical-shaped) metal nanoparticles have higher refractive index relative to UVB radiation and larger (e.g., coral-shaped) metal nanoparticles have higher refractive index relative to UVA radiation.

In some embodiments, sunscreen compositions can include metal nanoparticles and/or multi-component metal nanoparticles having a refractive index that is high enough with respect to UV radiation to protect against sunburns and other skin damage, but that is low enough with respect to visible light to avoid excessive scattering of visible light (e.g., resulting in a chalky, pasty, or otherwise opaque or semi-opaque appearance that may be undesirable in many sunscreen applications). For example, nanoparticles included in sunscreen compositions of the present disclosure can have a refractive index with respect to visible light (e.g., as measured at a standard 589 nm wavelength) ranging from about 1.0 to about 2.5, and more preferably from about 1.0 to about 2.0, and even more preferably from about 1.0 to about 1.5, or from about 1.0 to about 1.2, or about 1.0 (e.g., so as to be substantially transparent to visible light).

In some embodiments, the sunscreen compositions can include metal nanoparticles that are photostable such that when exposed to solar radiation (e.g., in an environment with a relatively high UV index of about 15), the nanoparticles and/or multi-component nanoparticles do not degrade or lose effectiveness in protecting against UV radiation (e.g., remain about 100% as effective, or remain about 95-100% as effective, or about 90-100% as effective, or about 80-100% as effective) over at least a given time period (e.g., about 1 hour, or about 2-4 hours, or about 4-6 hours, about 6-12 hours or longer, 1-3 days or longer, or even indefinitely).

Metal nanomaterials of the type disclosed herein and having diameters or sizes in the range of 10 to 40 nm have loose dielectric fields. When a large quantity of particles are relatively close together, the dielectric effect on light waves passing through is not attenuated but can be frequency-shifted either to the longer wavelength (red) end or to the shorter wavelength (blue) end of the electromagnetic spectrum. Sunscreen compositions that have a sufficient quantity of nanoparticles can affect UV wavelength and down-convert them to the longer wavelength (red) end of the spectrum to reduce entry of photonic energy at a level that reduces overall damage.

In some embodiments, sunscreen compositions can exhibit radiation protection properties. In some embodiments, compositions may include a plurality of nanoparticles (e.g., beryllium and/or gold) configured to absorb harmful radiation (e.g., alpha particles, beta particles, and/or gamma radiation), thereby reducing or eliminating an amount of radiation passing through the nanoparticle treated material.

In some embodiments, gold nanoparticles in sunscreen compositions down-convert incoming UV radiation into less harmful UV radiation. In some embodiments, gold nanoparticles may down-convert the wavelengths of incoming UV radiation by approximately 200 nm. In some embodiments, gold nanoparticles may down-convert incoming UV radiation from UV light to visible light. In some embodiments, gold nanoparticles may down-convert incoming UV radiation from UV wavelengths toward red and/or green wavelengths.

In some embodiments, gold nanoparticles may absorb incoming high energy UV radiation and down-convert to emit a lower energy wavelength, thereby imparting UV protection to the skin or other surface. Unexpectedly, the ability of the gold nanoparticles produced by methods described in the aforementioned Niedermeyer patents to perpetually down-convert wavelength/radiation energy does not deteriorate with use. That is, the gold nanoparticles retain their UV blocking or attenuating capabilities indefinitely and are not degraded by exposure to UV radiation. This beneficially prolongs the effectiveness of the sunscreen composition. This permits lower concentrations of gold nanoparticles or other wavelength-shifting metal nanoparticles to be used, resulting in compositions that are cheaper to produce while maintaining their integrity.

### Methods of Protecting Against UV Radiation

In some embodiments, a method of protecting skin from UV radiation comprises: (1) applying a sunscreen composition comprising a carrier and metal nanoparticles onto a treatment area, and (2) the sunscreen composition reflecting and/or down-converting at least a portion of the UV light incident upon the treatment area.

In some embodiments, a method of protecting against UV radiation comprises: (1) applying a sunscreen composition onto a treatment area, the sunscreen composition comprising (i) about 250 ppb to about 25 ppm of spherical-shaped metal nanoparticles having a particle size of about 8 nm or less, or about 1 nm to about 7 nm, or about 2 nm to about 6.5 nm, or about 3 nm to about 6 nm, (ii) about 250 ppb to about 25 ppm of coral-shaped metal nanoparticles having a particle size between 40 and 100 nm, and (iii) a milli-molar or micro-molar concentration of a stabilizing agent (e.g., grape seed extract), and (2) the sunscreen composition reflecting, scattering, blocking and/or down-converting UV radiation incident upon the treatment area.

In some embodiments, a method of protecting against UV radiation comprises: (1) applying a sunscreen composition comprising an aqueous solution onto a treatment area, the sunscreen composition comprising (i) about 500 ppb to about 50 ppm of spherical-shaped metal nanoparticles having a particle size of about 8 nm or less, or about 3 nm to about 14 nm, or about 5 nm to about 13 nm, or about 7 nm to about 12 nm, or about 8 nm to about 10 nm, (ii) about 250 ppb to about 25 ppm of coral-shaped metal nanoparticles having a particle size between 40 and 100 nm, and (iii) a milli-molar or micro-molar concentration of a stabilizing agent (e.g., grape seed extract), and (2) the sunscreen composition reflecting, scattering, blocking, and/or down-converting UV radiation incident upon the treatment area.

In some embodiments, a method of protecting against UV radiation comprises: (1) applying a sunscreen composition comprising an aqueous solution onto a treatment area, the sunscreen composition comprising (i) about 1 ppm to about 100 ppm of spherical-shaped metal nanoparticles having a particle size of about 9 nm to about 20 nm, or about 10 nm to about 18 nm, or about 11 nm to about 16 nm, or about 12 nm to about 15 nm, (ii) between about 250 ppb to about 50 ppm of coral-shaped metal nanoparticles having a particle size between 40 and 100 nm, and (iii) a milli-molar or micro-molar concentration of a stabilizing agent (e.g., grape seed extract), and (2) the sunscreen composition reflecting, scattering, blocking, and/or down-converting UV radiation incident upon the treatment area.

Other embodiments may contain spherical-shaped nanoparticles without coral-shaped nanoparticles or coral-shaped nanoparticles without spherical-shaped nanoparticles. In such embodiments, the spherical-shaped nanoparticles or the coral-shaped nanoparticles can be of substantially uniform size or can have different sizes (e.g., two or more sets of differently sized nanoparticles).

### Methods of Manufacture

In some embodiments, manufacturing sunscreen nanoparticle compositions having multi-component nanoparticles may comprise manufacturing first and second nanoparticle components (e.g., in embodiments including two separate nanoparticle components) in liquids that are compatible with the final composition.

For example, in the case of a water, alcohol, or water and alcohol based composition, both the first and second nanoparticle components can be manufactured in a water, alcohol, or water and alcohol based solution, the stabilizing agent can then be then added to one or both of the nanoparticle components, and the nanoparticle components can then be combined to yield desired concentrations.

In another example, such as in embodiments having a lotion or cream-based carrier, the first and second nanoparticle components can be either manufactured into one of the major components of the final composition or made in a water or alcohol (or water-alcohol mixture) and diluted into the lotion or cream based composition.

For example, stearic acid, oils, emulsifying wax, and other minor components may be heated to between 160° F. and 200° F. in order to create the desired final composition. After this nearly completed cream composition has cooled to less than about 105° F., first and second sets of nanoparticles, which have preferably been manufactured into a natural-based polyphenol or other stabilizing agent, can then be added to complete the final lotion or cream composition.

In some embodiments, coral-shaped nanoparticles are manufactured in one of the major components of the final composition. Spherical-shaped nanoparticles can then be added to the component containing the coral-shaped nanoparticles. In this way, a network created by the coral-shaped nanoparticles can receive, attract and/or hold the spherical-shaped nanoparticles. In some embodiments, the spherical-shaped nanoparticles comprise gold nanoparticles having a particle size less than 40 nm and that can down-convert incident UV radiation.

### EXAMPLES

### Example 1

A sunscreen composition is prepared and includes a solution of 90% water and 10% isopropyl alcohol having (i) 2 ppm of spherical-shaped gold (Au) nanoparticles having a mean diameter of 10 nm, with 99% of the Au nanoparticles having a diameter within +1 nm of the mean diameter, and (ii) grape seed extract at 1 milli molar concentration. The sunscreen composition can be readily applied to any region where protection from solar radiation, particularly UV radiation, is desired.

### Example 2

A sunscreen composition is prepared and includes a solution of 90% water and 10% isopropyl alcohol having (i) 1 ppm coral-shaped gold (Au) nanoparticles having a mean length of 40 nm, with 99% of the Au nanoparticles having a length (or cross section) within +6 nm of the mean length, (ii) 2 ppm of spherical-shaped gold (Au) nanoparticles having a mean diameter of 10 nm, with 99% of the Au nanoparticles having a diameter within +1 nm of the mean diameter, and (iii) grape seed extract at 1 milli molar concentration. The sunscreen composition can be readily applied to any region where protection from solar radiation, particularly UV radiation, is desired.

### Example 3

A sunscreen composition is prepared and includes a solution of 50% water and 50% isopropyl alcohol having (i) 0.5 ppm of spherical-shaped zinc (Zn) nanoparticles having a mean diameter of 10 nm, with 99% of the Zn nanoparticles having a diameter within +1 nm of the mean diameter, (ii) 1 ppm of spherical-shaped silver (Ag) nanoparticles having a mean diameter of 8 nm, with 99% of the Ag nanoparticles having a diameter within +1 nm of the mean diameter, and (iii) arjuna bark extract at 1 micro molar concentration. The sunscreen composition can be readily applied to any region where protection from solar radiation, particularly UV radiation, is desired.

### Example 4

A sunscreen composition is prepared and includes a solution of 90% water and 10% isopropyl alcohol having (i) 1 ppm coral-shaped zinc (Zn) nanoparticles having a mean length of 40 nm, with 99% of the Zn nanoparticles having a length (or cross section) within +6 nm of the mean length, (ii) 2 ppm of spherical-shaped silver (Ag) nanoparticles having a mean diameter of 10 nm, with 99% of the Ag nanoparticles having a diameter within +1 nm of the mean diameter, and (iii) grape seed extract at 1 milli molar concentration. The sunscreen composition can be readily applied to any region where protection from solar radiation, particularly UV radiation, is desired.

### Example 5

A sunscreen composition is prepared and includes a 30% water 70% isopropyl alcohol solution having (i) 3 ppm coral-shaped zinc (Zn) nanoparticles having a mean length of 80 nm, with 99% of the Zn nanoparticles having a cross section within +10 nm of the mean length, (ii) 5 ppm of spherical-shaped silver (Ag) nanoparticles having a mean diameter of 15 nm, with 99% of the Ag nanoparticles having a diameter within +1.5 nm of the mean diameter, and (iii) 1 millimolar concentration of an anionic detergent. The sunscreen composition is readily applied to any region where protection from solar radiation, particularly UV radiation, is desired.

### Example 6

A cream based carrier is prepared by heating stearic acid, olive oil, and emulsifying wax to between 160° F. and 200° F. Metal nanoparticles are added after cooling the composition to about 105° F.

### Example 7

A sunscreen composition is prepared by adding to the cream carrier of Example 4 (i) 1 ppm of coral-shaped zinc (Zn) nanoparticles having a mean length of 80 nm, with 99% of the Zn nanoparticles having a length within +10 nm of the mean length and (ii) 2 ppm of spherical-shaped titanium (Ti) nanoparticles having a mean diameter of 15 nm, with 99% of the Ti nanoparticles having a diameter within +1.5 nm of the mean diameter and (iii) 1 millimolar concentration of grape seed oil into which both the Ti and Zn nanoparticles are added before the grape seed oil is added to form the final product. This sunscreen composition can be readily applied to any region where protection from solar radiation, particularly UV radiation, is desired.

### Example 8

A sunscreen composition is prepared by adding to the cream carrier of Example 4 (i) 3 ppm of coral-shaped titanium (Ti) nanoparticles having a mean length of 80 nm, with 99% of the Ti nanoparticles having a length within +10 nm of the mean length and (ii) 5 ppm of spherical-shaped zinc (Zn) nanoparticles having a mean diameter of 10 nm, with 99% of the Zn nanoparticles having a diameter within +1 nm of the mean diameter and (iii) 1 millimolar concentration of grape seed oil into which both the Ti and Zn nanoparticles are added before the grape seed oil is added to the final product. This sunscreen composition can be readily applied to any region where protection from solar radiation, particularly UV radiation, is desired.

### Example 9

A sunscreen composition is prepared by adding to the cream carrier of Example 4 (i) 3 ppm of coral-shaped zinc (Zn) nanoparticles having a mean length of 80 nm, with 99% of these Zn nanoparticles having a length within +10 nm of the mean length, (ii) 3 ppm of spherical-shaped zinc (Zn) nanoparticles having a mean diameter of 10 nm, with 99% of these Zn nanoparticles having a diameter within +1 nm of the mean diameter, and (iii) 3 ppm of spherical-shaped zinc nanoparticles having a mean diameter of 15 nm, with 99% of the Zn nanoparticles having a diameter within +1.5 nm of the mean diameter, and (iv) 1 millimolar concentration of arjuna bark extract into which the nanoparticles are added before the arjuna bark extract is added to the final product. This sunscreen composition can be readily applied to any region where protection from solar radiation, particularly UV radiation, is desired.

### Example 10

A sunscreen composition is prepared and includes a solution of 50% water and 50% ethanol having (i) 0.5 ppm of spherical-shaped titanium (Ti) nanoparticles having a mean diameter of 10 nm, with 99% of the Ti nanoparticles having a diameter within +1 nm of the mean diameter, (ii) 1 ppm of spherical-shaped silver (Ag) nanoparticles having a mean diameter of 8 nm, with 99% of the Ag nanoparticles having a diameter within +1 nm of the mean diameter, (iii) 10 ppm of arjuna bark extract, and (iv) 10 ppm of aloe. This sunscreen composition can be readily applied to any region where protection from solar radiation, particularly UV radiation, is desired.

### Example 11

A sunscreen composition is prepared and includes a solution of 30% water and 70% ethanol having (i) 1 ppm coral-shaped zinc (Zn) nanoparticles having a mean length of 40 nm, with 99% of the Zn nanoparticles having a length within +6 nm of the mean length, (ii) 2 ppm of spherical-shaped silver (Ag) nanoparticles having a mean diameter of 10 nm, with 99% of the Ag nanoparticles having a diameter within +1 nm of the mean diameter, (iii) 30 ppm arjuna bark extract, and (iv) 30 ppm aloe. The sunscreen composition can be readily applied to any region where protection from solar radiation, particularly UV radiation, is desired.

### Example 12

A sunscreen composition is prepared and includes a solution of 70% water and 30% ethanol having (i) 3 ppm of coral-shaped zinc (Zn) nanoparticles having a mean length of 80 nm, with 99% of the Zn nanoparticles having a length within +10 nm of the mean length, (ii) 5 ppm of spherical-shaped silver (Ag) nanoparticles having a mean diameter of 10 nm, with 99% of the Ag nanoparticles having a diameter within +1 nm of the mean diameter, (iii) 30 ppm grape seed extract, and (iv) 20 ppm aloe. The sunscreen composition can be readily applied to any region where protection from solar radiation, particularly UV radiation, is desired.

### Example 13

A sunscreen composition is prepared and includes a 80% water 20% ethanol solution having (i) 3 ppm of coral-shaped zinc (Zn) nanoparticles having a mean length of 80 nm, with 99% of the Zn nanoparticles having a length within +10 nm of the mean length, (ii) 5 ppm of spherical-shaped silver (Ag) nanoparticles having a mean diameter of 10 nm. with 99% of the Ag nanoparticles having a diameter within +1 nm of the mean diameter, and (iii) 20 ppm grape seed extract. The sunscreen composition can be readily applied to any region where protection from solar radiation, particularly UV radiation, is desired.

### Example 14

A lotion carrier suitable for carrying a multicomponent antimicrobial composition is prepared by heating stearic acid, glycerin, coconut oil, and emulsifying wax to between 160° F. and 200° F. Nanoparticles are added after cooling the composition to about 105° F.

### Example 15

A sunscreen composition is prepared by adding to the lotion carrier of Example 12 (i) 1 ppm of coral-shaped zinc (Zn) nanoparticles having a mean length of 80 nm, with 99% of the Zn nanoparticles having a length within +10 nm of the mean length, (ii) 2 ppm of spherical-shaped silver (Ag) nanoparticles having a mean diameter of 15 nm, with 99% of the Ag nanoparticles having a diameter within +1.5 nm of the mean diameter, and (iii) 10 ppm grape seed oil into which both the Ag and Zn nanoparticles are added before the grape seed oil is added to the final product. The sunscreen composition can be readily applied to any region where protection from solar radiation, particularly UV radiation, is desired.

### Example 16

A sunscreen composition is prepared by adding to the cream carrier of Example 12 (i) 3 ppm of coral-shaped zinc (Zn) nanoparticles having a mean length of 80 nm, with 99% of these Zn nanoparticles having a length within +10 nm of the mean length, (ii) 5 ppm of spherical-shaped silver (Ag) nanoparticles having a mean diameter of 10 nm, with 99% of the Ag nanoparticles having a diameter within +1 nm of the mean diameter, and (iii) 20 ppm grape seed oil into which both the Ag and Zn nanoparticles are added before the grape seed oil is added to the final product. The sunscreen composition can be readily applied to any region where protection from solar radiation, particularly UV radiation, is desired.

### Additional Terms & Definitions

While certain embodiments of the present disclosure have been described in detail, with reference to specific configurations, parameters, components, elements, etcetera, the descriptions are illustrative and are not to be construed as limiting the scope of the claimed invention.

Furthermore, it should be understood that for any given element of component of a described embodiment, any of the possible alternatives listed for that element or component may generally be used individually or in combination with one another, unless implicitly or explicitly stated otherwise.

In addition, unless otherwise indicated, numbers expressing quantities, constituents, distances, or other measurements used in the specification and claims are to be understood as optionally being modified by the term “about” or its synonyms. When the terms “about,” “approximately,” “substantially,” or the like are used in conjunction with a stated amount, value, or condition, it may be taken to mean an amount, value or condition that deviates by less than 20%, less than 10%, less than 5%, less than 1%, less than 0.1%, or less than 0.01% of the stated amount, value, or condition. At the very least, and not as an attempt to limit the application of the doctrine of equivalents to the scope of the claims, each numerical parameter should be construed in light of the number of reported significant digits and by applying ordinary rounding techniques.

Any headings and subheadings used herein are for organizational purposes only and are not meant to be used to limit the scope of the description or the claims.

It will also be noted that, as used in this specification and the appended claims, the singular forms “a,” “an” and “the” do not exclude plural referents unless the context clearly dictates otherwise. Thus, for example, an embodiment referencing a singular referent (e.g., “widget”) may also include two or more such referents.

It will also be appreciated that embodiments described herein may also include properties and/or features (e.g., ingredients, components, members, elements, parts, and/or portions) described in one or more separate embodiments and are not necessarily limited strictly to the features expressly described for that particular embodiment. Accordingly, the various features of a given embodiment can be combined with and/or incorporated into other embodiments of the present disclosure. Thus, disclosure of certain features relative to a specific embodiment of the present disclosure should not be construed as limiting application or inclusion of said features to the specific embodiment. Rather, it will be appreciated that other embodiments can also include such features.

The present invention may be embodied in other specific forms without departing from its spirit or essential characteristics. The described embodiments are to be considered in all respects only as illustrative and not restrictive. The scope of the invention is, therefore, indicated by the appended claims rather than by the foregoing description. All changes which come within the meaning and range of equivalency of the claims are to be embraced within their scope.

1. A sunscreen composition for protecting against ultraviolet radiation, the sunscreen composition comprising:
a carrier that is readily applied to an area of a body; and
a plurality of nonionic metal nanoparticles suspended in the carrier and configured in size and shape to selectively scatter and/or down-convert ultraviolet (UV) radiation incident upon the nanoparticles.

2. The sunscreen composition of claim 1, wherein the metal nanoparticles selectively scatter UV radiation by having a higher refractive index with respect to UV radiation than a refractive index with respect to visible light.

3. The sunscreen composition of claim 2, wherein the refractive index of the metal nanoparticles with respect to UV radiation is about 1.5 to about 4.6, about 2.0 to about 4.0, or about 2.5 to about 3.5.

4. The sunscreen composition of claim 2, wherein the refractive index of the metal nanoparticles with respect to UV radiation is higher with respect to UVB radiation than to UVA radiation.

5. The sunscreen composition of claim 2, wherein the refractive index of the metal nanoparticles with respect to UV radiation is lower with respect to UVB radiation than to UVA radiation.

6. The sunscreen composition of claim 2, wherein the refractive index of the metal nanoparticles with respect to visible light is about 1.0 to about 2.5, about 1.0 to about 2.0, about 1.0 to about 1.5, or about 1.0.

7. The sunscreen composition of claim 1, wherein the metal nanoparticles have a photostability that provides maintained effectiveness for a time period of at least about 6 hours in a high ultraviolet radiation environment.

8. The sunscreen composition of claim 7, wherein the maintained effectiveness is about 80-100%, about 90-100%, or about 99-100%.

9. The sunscreen composition of claim 1, wherein the metal nanoparticles include spherical-shaped metal nanoparticles and a stabilizing agent.

10. The sunscreen composition of claim 9, wherein the spherical-shaped metal nanoparticles provide broad spectrum protection against UVA and UVB radiation.

11. The sunscreen composition of claim 9, wherein the spherical-shaped metal nanoparticles have a particle size of about 4 nm to about 20 nm, or about 6 nm to about 15 nm, or about 8 nm to about 12 nm.

12. The sunscreen composition of claim 9, wherein the spherical-shaped metal nanoparticles have a sphericity of at least 0.99.

13. The sunscreen composition of claim 9, wherein the stabilizing agent is a plant-based extract.

14. The sunscreen composition of claim 9, wherein the stabilizing agent comprises at least one of grape seed extract, arjuna bark, or witch hazel.

15. The sunscreen composition of claim 9, further comprising coral-shaped metal nanoparticles.

16. The sunscreen composition of claim 15, wherein the coral-shaped metal nanoparticles have a diameter ranging from about 15 nm to about 100 nm, or about 25 nm to about 95 nm, or about 40 nm to about 90 nm, or about 60 nm to about 85 nm, or about 70 nm to about 80 nm.

17. The sunscreen composition of claim 1, wherein the carrier is a cream or lotion that includes one or more of glycerin or stearic acid.

18. The sunscreen composition of claim 17, wherein the cream or lotion further includes one or more oils selected from the group consisting of coconut oil, olive oil, grape seed oil, shea butter, mango butter, and Vitamin E oil.

19. The sunscreen composition of claim 1, wherein the sunscreen composition comprises about 250 ppb to about 100 ppm of metal nanoparticles by weight, or about 500 ppb to about 50 ppm, or about 1 ppm to about 25 ppm, or about 3 ppm to about 20 ppm metal nanoparticles by weight.

20. A method of protecting against ultraviolet radiation, the method comprising:
applying a sunscreen composition according to claim 1 to a treatment area; and
the sunscreen composition reflecting and/or down-converting at least a portion of the UV radiation incident upon the treatment area.

21. A method of manufacturing a sunscreen composition according to claim 1, the method comprising:
suspending a plurality of nonionic metal nanoparticles in a carrier,
wherein the plurality of nonionic metal nanoparticles are configured in size and shape to selectively scatter and/or down-convert UV radiation incident upon the nanoparticles, and
wherein the carrier is formulated for ready application to a treatment area of a body.
